(19) United States
(12) Patent Application Publication
(10) Pub. No.: US 2021/0087232 A1
(43) Pub. Date: Mar. 25, 2021

(54) D-ENANTIOMERIC PEPTIDES FOR ANTI-INFLAMMATORY TREATMENT OF AMYOTROPIC LATERAL SCLEROSIS (ALS) AND OTHER DISEASES DRIVEN BY NEURO-INFLAMMATION

(71) Applicant: FORSCHUNGSZENTRUM JUELICH GMBH, Juelich (DE)

(72) Inventors: Janine KUTZSCHE, Dueren (DE); Dieter WILLBOLD, Juelich (DE)

(21) Appl. No.: 17/052,258

(22) PCT Filed: May 2, 2019
(86) PCT No.: PCT/EP2019/061298

§ 371 Date: Nov. 2, 2020

(30) Foreign Application Priority Data
May 3, 2018 (DE) 10 2018 110 543.6

(51) Int. Cl. C07K 7/08

(52) U.S. Cl. CPC C07K 7/08; A61K 38/00

(57) ABSTRACT

The present invention relates to a polymer comprising at least one monomer, the at least one monomer comprising a peptide having an amino acid sequence selected from the group consisting of SEQ ID NO: 1 or homologs with an identity of at least 80% thereof, and a pharmaceutical composition comprising such a peptide for use in the treatment of amyotrophic lateral sclerosis (ALS).

The present invention relates to a polymer and a pharmaceutical composition comprising such a polymer for use in the treatment of amyotrophic lateral sclerosis.

Amyotrophic lateral sclerosis, ALS for short, is a neurodegenerative disease of the motor nervous system that is characterized by progressive, irreversible loss of motor neurons in middle age. With an incidence of 2 per 100 000, ALS is a rare disease. The clinical picture of ALS is characterized by peripheral and central pareses which are caused by damage or degeneration of the upper and lower motor neurones. In the course of the disease, initial asymmetric paralysis in the extremities and musculature for speech and swallowing is usually followed relatively rapidly by a generalization of the paresis. The survival time of patients is generally 3 to 5 years after the appearance of the initial symptoms. More recent findings indicate that non-neuronal cells also play a role in ALS symptoms by, for example, activating inflammatory processes and/or influencing signal transduction processes.

To date, there is no causal therapy of ALS. The only medicament currently authorized, riluzole, a sodium channel blocker which attenuates the effect of the neurotransmitter glutamate, is only capable of prolonging the survival time of patients by a few months.

It is thus of utmost urgency to provide an active ingredient for causal therapy of ALS that is capable of healing or at least significantly slowing the disease.

The suppression of inflammatory reactions appears to play an important role in this connection. However, hitherto known anti-inflammatories are a failure, such as acetylsalicylic acid or nonsteroidal anti-inflammatories, such as, for example, ibuprofen, which act via the inhibition of the enzymes COX1 and COX2. For this reason, there is an increased search for anti-inflammatories which do not act via the inhibition of COX1 and/or COX2.

It is an object of the present invention to eliminate the abovementioned disadvantages of the prior art. In particular, it is an object of the present invention to provide a substance which is capable of causally therapying ALS or at least distinctly slowing the course of the disease.

The present invention achieves this by the use of a polymer according to claim 1, especially by a polymer comprising at least one monomer, the at least one monomer comprising a peptide having an amino acid sequence selected from the group consisting of SEQ ID NO: 1 ptlhthnrrrrr or homologs with an identity of at least 80%, thereof preferably amidated at the C-terminus, in the treatment of amyotrophic lateral sclerosis (ALS).

In the context of the present invention, the term “polymer” encompasses all “multimers” comprising at least two monomer units. Thus, the term “polymers” in the context of the present invention also encompasses, for example, dimers, trimers and oligomers.

In the context of the present invention, the term “comprise” can also mean “consisting of”.

In the context of the invention, “homologous sequences” or “homologs” means that an amino acid sequence has an identity of at least 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100% with one of the abovementioned amino acid sequences of the monomers. In the present description, the terms “homologous” or “homology” are used synonymously instead of the term “identity”. The identity between two nucleic acid sequences or polypeptide sequences is calculated by comparison with the aid of the program BESTFIT based on the algorithm by Smith, T. F. and Waterman, M. S. (Adv. Appl. Math. 2: 482-489 (1981)) with setting of the following parameters for amino acids: gap creation penalty: 8 and gap extension penalty: 2; and of the following parameters for nucleic acids: gap creation penalty: 50 and gap extension penalty: 3.

Preferably, the identity between two nucleic acid sequences or polypeptide sequences is defined by the identity of the nucleic acid sequence/polypeptide sequence over the entire sequence length in each case, as calculated by comparison with the aid of the program GAP based on the algorithm by Needleman, S. B. and Wunsch, C. D. (J. Mol. Biol. 48: 443-453) with setting of the following parameters for amino acids: gap creation penalty: 8 and gap extension penalty: 2; and the following parameters for nucleic acids: gap creation penalty: 50 and gap extension penalty: 3.

In the context of the present invention, two amino acid sequences are identical if they have the same amino acid sequence.

In a further variant, homologs are also to be understood to mean the corresponding retro-inverso sequences of the abovementioned monomers. According to the invention, the term “retro-inverso sequence” refers to an amino acid sequence which is composed of amino acids in enantiomeric form (inverso: chirality of the alpha-carbon atom inverted) and in which additionally the order of the sequence has been reversed in relation to the original amino acid sequence (retro =backwards).

The polymer according to the invention is furthermore preferably characterized in that the at least one monomer comprises a peptide having an amino acid sequence selected from the group consisting of SEQ ID NO: 1 (RD2 monomer) ptlhthnrrrrr or homologs with an identity of at least 90%, 91%, 92%, 93%, 94%, 95%, 96%, 97%, 98%, 99%, 100%, thereof preferably amidated at the C-terminus.

Furthermore, sequences containing the abovementioned sequences are also used as monomers.

In a further variant, the monomers have fragments of the abovementioned sequences.

The polymer according to the invention is preferably characterized in that it contains 2, 3, 4, 5, 6, 7, 8, 9, 10 or more monomers, the polymer particularly preferably comprising 2 monomers.

The polymer according to the invention is preferably further characterized in that it contains identical or nonidentical monomers.

Particularly preferably, the polymer according to the invention comprises a dimer of two peptides of SEQ ID NO 1 (RD2).

In one embodiment of the invention, the monomers are covalently linked to one another. In a further embodiment, the monomers are not covalently connected to one another.

In the context of the invention, a covalent connection or linkage of the monomer units exists if the peptides are linked to one another in a linear manner head-to-head, tail-to-tail or head-to-tail.

At the same time, the monomers can be linked to one another directly or via a linker group. A linker is to be understood to mean one or more molecules which are bound to the monomers via covalent bonds, it being possible for said linkers to be linked to one another by covalent bonds too.

A noncovalent linkage in the context of the invention exists if the monomers are linked to one another via biotin and streptavidin, especially streptavidin tetramer.

In one variant of the present invention, the monomers can be linked to one another in a linear manner, especially as described above. In another variant, the monomers are linked to one another in a branched manner to form the polymer according to the invention.

According to the invention, a branched polymer can be a dendrimer in which the monomers are covalently or noncovalently linked to one another or that the monomers are linked to a platform molecule or are linked in a combination of these options.

Alternatively, combinations of these options are also possible.

Particularly preferably, the polymer according to the invention is a polymer which comprises two peptides of SEQ ID NO. 1 which are covalently linked to one another, yielding a peptide having amino acid SEQ ID NO: 2 (RD2RD2).

Very particularly preferably, the polymer according to the invention consists of a peptide of SEQ ID NO. 2 (RD2RD2) ptlhthnrrrrrptlhthnrrrrr.

Furthermore, it is particularly preferred that the polymer or peptide according to the invention consists of D-amino acids to a substantial or complete extent.

In the context of the present invention, the term “of D-amino acids to a substantial extent” means that the monomers to be used are built up from D-amino acids to an extent of at least 60%, preferably 75%, 80%, particularly preferably 85%, 90%, 95%, especially 96%, 97%, 98%, 99%, 100%.

The above-described polymers can, for example, be produced via chemical synthesis or peptide synthesis.

In a further preferred embodiment, the polymer is linked to a further substance.

In one embodiment, the invention also provides peptides according to the invention that are linked to a further substance. In the context of the invention, the linkage is a chemical bond as defined in Römpp Chemie Lexikon [Römpp's chemistry lexicon], 9th edition, volume 1, pages 650 ff., Georg Thieme Verlag Stuttgart, preferably a primary valency bond, especially a covalent bond.

In one variant, the substances are drugs or active ingredients, respectively defined in section 2 and section 4(19) of the Arzneimittelgesetz [German drugs act], as of September 2012. In one alternative, active ingredients are therapeutically active substances which are used as medicinally effective substances.

Another alternative concerns compounds which improve the solubility of the peptides, the absorption of the peptides in the gastrointestinal tract and/or crossing of the blood-brain-barrier.

It is preferred here if the further substance can be cleaved off by metabolization after administration in the body of the patient, returning the polymer according to the invention to a “free” state. Such systems are also known by the term “prodrugs”. Such prodrugs are of strategic importance especially in those cases in which the active ingredient that is actually active, for example the polymer according to the invention, does not reach the desired site of action or only reaches it to a negligible extent or does not reach it with sufficient selectivity if said active ingredient were administered in a direct manner. In this case, the prodrug concept mainly aims at improving pharmacokinetic properties of substances. The use of prodrugs can, for example, improve oral absorption or bioavailability, reduce the first-pass effect or enable a drug substance to cross the blood-brain barrier.

Sequences usable according to the invention are presented below:

SEQ ID NO 1:
ptIhthnrrrrr (RD2)
SEQ ID NO 2:
ptIhthnrrrrrptIhthnrrrrr (RD2RD2)

The present invention further provides a pharmaceutical composition comprising a polymer as defined above for use in the treatment of ALS.

Preferably present in such a composition is a polymer as defined above in a pharmaceutically effective concentration and also the customary additives.

The present invention will be elucidated below on the basis of some nonrestrictive examples.

### EXAMPLES

A polymer according to the invention in the form of a dimer of RD2 was prepared. The polymer according to the invention (RD2RD2) has SEQ ID NO: 2. The polymer according to the invention consisted exclusively of D-amino acids.

To analyze the anti-inflammatory effect of RD2RD2 as a potential therapeutic for treating ALS, two treatment studies were carried out in transgenic ALS mice (tg G93R SOD1 mice). With the aid of various behavioral and motor tests (open field test, SHIRPA test battery, pole test, hind-leg grip strength test, rotarod test, hind-leg spread test) and histological analyses, it was possible to show that RD2RD2 delays disease onset, slows disease progression and reduces inflammatory reactions.

1.-14. (canceled)

15. A method of treating amyotrophic lateral sclerosis (ALS), wherein the method comprises administering to a patient afflicted by ALS a polymer which comprises at least one monomer comprising a peptide having an amino acid sequence selected from SEQ ID NO: 1 and homologs with an identity of at least 80% thereof in an amount which is effective for treating ALS.

16. The method of claim 15, wherein the amino acid sequence selected from SEQ ID NO: 1 and homologs with an identity of at least 80% thereof is amidated.

17. The method of claim 15, wherein the at least one monomer comprises a peptide having an amino acid sequence selected from SEQ ID NO: 1 (RD2 monomer) and homologs with an identity of at least 90% thereof.

18. The method of claim 17, wherein the amino acid sequence selected from SEQ ID NO: 1 and homologs with an identity of at least 90% thereof is amidated.

19. The method of claim 15, wherein the peptide comprises from 2 to 10 monomers.

20. The method of claim 15, wherein the peptide comprises 2 monomers.

21. The method of claim 15, wherein the peptide comprises identical monomers.

22. The method of claim 15, wherein the peptide comprises non-identical monomers.

23. The method of claim 15, wherein the peptide comprises a dimer of two RD2 peptides.

24. The method of claim 15, wherein the polymer comprises or consists of a peptide having amino acid SEQ ID NO: 2 (RD2RD2).

25. The method of claim 15, wherein the peptide consists substantially of D-amino acids.

26. The method of claim 15, wherein the peptide is linked to a further substance.

27. The method of claim 15, wherein the monomers are covalently linked to one another.

28. The method of claim 15, wherein the monomers are non-covalently linked to one another.

29. The method of claim 15, wherein the monomers are linked to one another directly without a linker group.

30. The method of claim 15, wherein the monomers are linked to one another indirectly through a linker group.

31. The method of claim 15, wherein the monomers are linked to one another in a linear fashion.

32. The method of claim 15, wherein the monomers are linked to one another in a branched fashion.

33. The method of claim 15, wherein a dendrimer is involved or the monomers are linked to a platform molecule or are linked in a combination of these options.

34. A pharmaceutical composition for the treatment of ALS, wherein the composition comprises (i) a polymer which comprises at least one monomer comprising a peptide having an amino acid sequence selected from SEQ ID NO: 1 and homologs with an identity of at least 80% thereof and (ii) one or more pharmaceutically acceptable excipients.
